(19) United States
(12) Patent Application Publication
(10) Pub. No.: US 2003/0156970 A1
(43) Pub. Date: Aug. 21, 2003

(54) Sulphur-free lignin and derivatives thereof for reducing the formation of slime and deposits in industrial plants

(76) Inventors: Jorg Oberkofler, Salzburg (AT); Jeff Spedding, Salzburg (AT)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/221,735

(22) PCT Filed: Feb. 21, 2001
(86) PCT No.: PCT/EP01/01972

(30) Foreign Application Priority Data
Mar. 16, 2000 (DE) 100 12 894.7
Apr. 5, 2000 (DE) 100 17 012.9

(51) Int. Cl. A61L 2/16

(52) U.S. Cl. 422/7; 422/14; 422/28; 424/725

(57) ABSTRACT

The invention concerns a method for the reducing the formation of slime and deposits in industrial plants, in which water or aqueous process liquids are circulated, sulphur-free lignin or a derivative thereof being added to the system in a quantity proportional to the quantity of the slime and deposit-causing substances present in the water.

### DESCRIPTION

The present invention relates to a method for reducing slime and deposit formation in closed and/or partly closed aqueous or aquiferous systems. The process is applicable in aqueous solutions, emulsions and suspensions stored in storage tanks, industrial plants in which water is circulated, such as paper machines or cooling water circuits.

Micro-organisms of one form or another typically find ideal growing conditions in the water circuits of and/or tanks of industrial systems. The specific conditions present there influence the type of micro-organisms. The process water, and here in particular the white water, as it is known in paper machines, has been subject in recent years to increased recycling and circuit closure and these changes increasingly provide ideal conditions in the form of temperature, pH value, nutrients available etc. for extensive growth of micro-organisms. Many such organisms tend to form slime colonies, instead of forming individual, freely dispersed units, which leads to progressive growth of slime deposits in the circuit.

Moreover, various additives such as natural or synthetic polymers, fillers and other finely divided components used in paper making, also have a tendency to create deposits in the white water circuits. Clearly, the combination of microbiologically caused slime and these additional deposits can lead to even more serious deposit problems.

Furthermore it is also of significance that in a special way filtered or treated process water or even fresh water is often used on paper machines to create sprays or jets of water which are used, for example, to damp down foam, to prevent the build-up of deposits of paper fibres and fillers in critical regions or to determine the width of the wet paper sheet. In the interface regions between this spray water and the main white water circuits, where, for example, cold fresh water rich in oxygen can encounter hot process water rich in nutrients, specific conditions can prevail which are very conducive to the growth of micro-organisms, more especially of filamentous forms. The same applies in a similar way also to the mixing interfaces of different circuits.

The detachment of these deposits from walls and other structures in the circuit into the white water, either directly onto moving paper machine or even onto the paper sheet, leads to damage or holes in the paper and can cause the paper web to be torn which results in a machine down time.

Significant quantities of biocides are added to the white water in order to combat these deposit problems. Due to the diversity and the flexibility of the micro-organisms which include not only bacteria, but also fungi, the choice of biocide is not easy. Due to the adaptability of micro-organisms it is extremely difficult to reduce their growth to a ‘safe’ or trouble-free level. The only effective way to control microbial deposits with the help of biocides is the attempt to kill virtually all of these organisms. The complexity of the systems and also the necessary service controls mean that effective treatment is not cheap.

Of necessity and also often due to the statutory requirements biocides are normally degradable so that in the case of normal usage levels they do not cause any problems for the environment or for the function of downstream waste water and/or clarification plants. On the other hand, they present a potential danger in handling, transport and in accidental misuse.

For reasons of improved efficiency, environmental protection and for cost reasons it has long been an aim of the paper industry to reduce the use of biocides by using other methods.

A well-known method for the reduction of slime deposits is the addition of lignosulphonates for the paper machine white water. It is also used in combination with biocides, this permitting a reduction of the biocide quantity used. These methods are described in the European printed patent specifications Nos. 0 185 963 and 0 496 905.

The problem to be dealt with by the present invention is to increase the effectiveness of the slime and deposit control systems and at the same time to keep the effects on the environment at least at the same level or even to reduce them.

Surprisingly this problem was solved by adding, in accordance with claim 1 of the present application, sulphur-free lignin or a derivative thereof to the system in a quantity which is proportional to the quantity of slime-causing substances present in the water. Preferred embodiments can be seen from the claims 2 to 23.

In a comparison of soluble aromatic polymers derived from lignin or its derivatives, it did namely turn out that the sulphur-free lignin and the lignin derivatives which are used according to the invention are more effective than the currently used lignosulphonates in the prevention and reduction of slime and deposit formation in systems in which paper machine white water is circulated.

Moreover, the products used according to the invention, since they are essentially non-toxic, clearly have the required advantages for the environment in comparison with biocides.

In a process for the production of pulp fibres which is known as the ‘soda’ pulp process, the resultant liquid, what is referred to as the ‘black liquor’, contains substances from lignin degradation in the form of carboxylated lignin derivatives. A modified form of this method is known as the ‘Soda anthraquinone or soda AQ process’. These methods are very well suited to the processing of non-ligneous materials, typically annual plants such as straw, flax and hemp.

In a further process for the production of cleaned, soluble polymeric substances by temperature-dependent and pH-dependent precipitation—such as is described in the WO 89/00512 ‘Process for the treatment of alkaline solutions containing aromatic polymers’—specific fractions of carboxylated lignin derivatives can be produced. In this process the black liquor is brought to a relatively low temperature and acidified to a certain level at which the precipitation occurs. The acidifying typically goes as far as a pH value of approx. 1-2 units below the pH value of the point of change of the titration of the black liquor with an acid. The viscosity of the resultant mixture typically reaches a maximum at a pH value just below the point of change and is normally so high that the separation of the precipitated lignin by filtration or centrifuging is not commercially feasible. This mixture is, however, subsequently heated up to a certain temperature which depends on the type of black liquor, the viscosity of the liquid mixture at the same time decreasing so markedly that a successful separation of the carboxylated lignin is possible using industrially feasible methods.

By checking of the raw material and control of the process conditions both in the soda-anthraquinone process and also the precipitation of carboxylated lignin the quality and the properties of the carboxylated lignin derivatives can be influenced in a specific way.

In a further process, in which with the use of tetramethylammonium hydroxide (TMAH) as a boiling liquor vegetable material originating from wood is used for the production of pulp, sulphur-free lignin is obtained which can be cleaned by precipitation. This lignin is low-molecular and soluble in water in the alkaline range.

Lignosulphonates, including the commercial products which are offered for the purpose of slime control, have certain structural characteristics which consist of the fact that the sulphonate groups are located on the C3 side chain of the molecular phenylpropane basic unit of the lignin. The commercial lignosulphonate slime combating products examined show a sulphur content of approx. 5-6%. The share of carboxyl groups of commercial lignosulphonates is normally not shown in the relevant literature, but it is known that they are difficult to clean and that they can contain uronic acid. This means that any carboxyl groups possibly present do not have to be linked with the basic lignin unit. The localising of any carboxyl groups within a lignosulphonate unit is, according to the literature, not really clear. It was proposed that they should be located on the C1 or C5 positions of the ring. According to Sarkanen (Sarkanen and Ludwig, ‘Lignins’, John Wiley & Sons, 1971) the evidence for this is, however, not very convincing.

Carboxylated lignin derivatives of annual plants which are used according to the invention typically have a carboxyl content of 5-7% and a sulphur content of typically less than 0.05%. If one follows the literature on the subject, the —COOH groups in lignin from grasses, for example, stem from coumarinic acid groups and to a smaller percentage from feruleinic acid groups (Shimada, Fukuzuka and Higuchi, Tappi 54 (1) 72-78 (1971)). That suggests that the carboxyl groups are located on aliphatic side chains and not on the ring.

It is therefore clear that carboxylated lignin which is referred to in the method of this invention shows a different molecular structure and different chemical composition than the lignosulphonate, and that these differences must be responsible for the different performances of these products.

Upon the addition of a defined quantity of sodium hydroxide the carboxylated lignin derivatives become water-soluble; with a sufficient quantity of alkali, typically 4-10% of NaOH based on carboxylated lignin, for example a 33% solution has a viscosity of 500 cPs at pH 9 and 40 cPs at pH 12, a product thus being produced which combines a content of active substances suitable for commercial use with a viscosity which permits simple dosing and subsequent mixing with the white water to be treated.

The alkali-soluble lignin used here, which is obtained by the treatment of wood with TMAH, has a molecular weight of 1000 or slightly higher and is essentially sulphur-free. From this it becomes clear that this lignin likewise shows a different chemical structure and composition from those of the lignosulphonates and that these differences must be responsible for the different performances of these products. The exact structure of this type of lignin has not yet been adequately studied, but it is known that a certain degree of carboxylation is present, and it can be proposed that the basic structures of the carboxylated lignin from the soda process carried out on annual plant material and the TMAH lignin from wood are not dissimilar, but both differ markedly from that of lignosulphonates.

Although within the scope of the present patent application mention is always made only of sulphur-free lignin or sulphur-free, carboxylated lignin, it is to be stated that an important factor for the efficiency of the products is their water-solubility. It is thus conceivable that also other water-soluble lignin derivatives which are sulphur-free could be used according to the invention, e.g. phosphonated or nitrated products.

In the case of certain types of paper containing more fillers, especially if the filler is a type of calcium carbonate, the paper machine cycle can be more prone to the formation of deposits, these deposits—whether originally caused by microbiological activity or chemical conditions being an open question—being particularly rich in carbonate, often also in combination with material of biological origin.

Surprisingly it turned out that simultaneous use of certain complexing agents together with the sulphur-free lignin or the lignin derivatives from this invention—added either separately or as a mixture to the paper machine white water—significantly improves the prevention or the reduction of slime and deposits forming in the white water circuit.

To be more precise, a solution of polyaspartic acid (—Na salt), mixed as a smaller percentage in a dissolved mixture with the carboxylated lignin of this invention, yielded significantly good results for the treatment of slime and deposits. The advantage of polyasparaginate in comparison with other available complexing agents, e.g. polyacrylates, is that it has a high degree of biological degradability, a fact which is an ecological advantage.

In this way the product under this invention can be produced specifically tailored to the requirements of the particular problem.

For the production of cellulose fibres from non-wood-containing sources such as straw, hemp or flax, long, strong fibres are produced which are advantageous for certain types of paper. The resultant black liquor from the production of fibres, including the soda AQ process, does, however, normally contain large quantities of silica. This means that the conventional disposal of black liquor containing lignin, e.g. by concentration and combustion as a fuel is not possible, so that the waste is often disposed of with negative consequences for the environment. The use of black liquor from the soda AQ process for the production of the product under this invention therefore yields additional clear advantages for the environment.

Moreover, as a result of the soda AQ process no sulphurous waste water or gases are produced which likewise entails a marked advantage for the environment as compared with some other methods of fibre production.

Used in accordance with the method of this invention, the sulphur-free lignin or the lignin derivatives, such as carboxylated lignin, can be used with added quantities (based on the active product content) of typically 0.1 g/m³ to 1.8 g{m³ of circuit water or paper machine white water. This usually results also in 10 g/t to 180 g/t, based on the solids content of the fluid containing the paper stock, if the solids content of the low-density stock in the stock flow onto the wire is approximately 1%. The carboxylated lignin is added continuously over 24 hours as a concentrated solution directly to the white water of a paper machine.

If, for example, for paper stock with a high percentage of carbonate filler additionally a complexing agent, e.g. polyasparaginate, is mixed with the sulphur-free lignin, the quantity of active complexing agent, calculated as a percentage share of the sulphur-free lignin which is present in the product liquid, can be typically 5-25%, preferably 10 to 20%, a particularly effective concentration being 15%. This mixture is likewise continuously added to the white water.

The composition of paper stock usually includes the addition of recycling paper or rejects which can be stored for some time as wet, concentrated stock, or also of process water which is stored under semi-static conditions. Reprocessed rejects can make up 20-30% of the stock used for paper production. Such stocks can, not only due to the storage conditions, be prone to biological degradation, but also due to large quantities of nutrients such as, for example, starch, and can be a significant source of contamination of the main white water circuits in paper machines. In such cases biocides can be used in order to preserve the rejects during storage, which is the usual practice. In relation to the process of this invention small quantities of a suitable biocide can be added to paper stocks or to the white water in order to supplement the effect of the sulphur-free lignin. Typically the biocide level use is much lower than in white water circuits which are only treated with biocide against slime deposits.

If paper stocks for the production of which, for example, rejects potentially contaminated by microbes, are to be treated, and—as a part of the method of this invention—a slight addition of biocide is to be made in addition to the sulphur-free lignin, a quantity of 10 to 180 g/t or more, typically 50-100 g/t, based on the solids content of the fluid containing the paper stock, can be added. A quantity of 70 g/t proved to be particularly effective. The biocide can be added periodically, not continuously at a place at which high-density stocks are mixed, or directly into the white water circuit. The times during which biocide is added can typically amount to 10-30% of the whole treatment period, for example one hour of biocide addition every six hours, the sulphur-free lignin being added continuously over 24 hours. Among the biocides which in combination with the sulphur-free lignin show good effectiveness in the method according to this invention, are 3,5-dimethyl-tetrahydro-1,3,5-thiadiazin-2-thion, methylene-bis-thiocyanate, carbamates, 2,2-dibromium-3-nitrile-propionamide and 2-bromium-2-nitropropane-1,3-diol. The biocide 2-bromium-2-nitropropane-1,3-diol which can be used as 20% solution, is as an additional biocide particularly effective in the case of a dosage of, for example, 70 g/t. Biocides with an inhibitory effect sustained for a longer period have proved more effective in combination with sulphur-free lignin than combinations with short-lived ‘killer’ or sterilising biocides.

In order to be bale to quantify the effects of various systems for slime and deposit control, a small test unit, as can be seen from diagram 1, was used. That unit is, for example, supplied with fresh paper machine white water. The central circuit is filled with white water and this is circulated. The temperature is set in such a way it corresponds to that in a typical paper machine. Fresh water or clear filtrate can be used for dilution and specifically as spray water. In an adjoining vessel at least two slime measuring boards are exposed to the circuit water, one of them remaining fully immersed and the other one half protruding from the water. The board half in the air can at certain temperatures be continuously sprayed with fresh water or process water, in order to simulate the spray water areas in the paper machines. Because of these additions a similar volume overflows into the drain. The whole unit can, if necessary, be set up near a paper machine. All of the parameters such as temperature, pH value, dissolved oxygen, flow, addition of additives, intensity of the incident light, humidity etc. can be checked and/or measured.

The slime measuring boards are of a known weight and can be periodically checked and weighed in order to measure the growth of the deposits. The board can also be dried and then weighed in order to measure the dry weight of the slime or to determine the chemical composition.

The sulphur-free lignin or its derivatives such as carboxylated lignin and all additional complexing agents or biocides as they are provided for in this invention, can be dosed in suitable quantities and the effects can be measured. In this way it has been possible to show that the sulphur-free lignin or its derivatives is able to successfully solve the problem which is at the basis of this invention.

### EXAMPLES

Products and Processes for Their Production:

### Example 1

### Production of Black Liquor Containing Sulphur-Free Carboxylated Lignin Derivatives, From Wheat Straw Using a Soda AQ Process

Wheat straw was boiled for one hour at 160° C. in a pressure reactor with 16% NaOH and 0.5% anthraquinone—based on dry weight of the plant material. During cooling-down the stock obtained was washed with a minimum of water. The black liquor was retained for further treatment in order to separate a fraction of carboxylated lignin derivatives which were intended for use in the method of this invention.

### Example 2

### Production of Black Liquor Containing Sulphur-Free Carboxylated Lignin Derivatives From Flax and Hemp Straw Using a Soda AQ Process

A mixture of flax and hemp straw was boiled for one hour at 160° C. in a pressure reactor together with 16% NaOH and 0.5% anthraquinone—based on the dry weight of the plant material. During cooling down the stock obtained was washed with a minimum of water. The black liquor was retained for further treatment in order to separate off a fraction of carboxylated lignin derivatives which were intended for use in the method of this invention.

### Example 3

### Production of Sulphur-Free Carboxylated Lignin From Wheat-Straw Black Liquor by Means of a Precipitation Process

Black liquor from wheat-straw pulp which was produced using the soda AQ process in example 1 was used. The liquor had an organic-substance content, measured as COD (chemical oxygen demand), of 120 g/l, a total basicity, expressed as g/l of NaOH, of 18 g/l, a point of change of the acid titration curve at pH 3 and the maximum viscosity with progressive acidifying at pH 2. The black liquor was heated up to 85° C. and acidified with sulphuric acid up to a pH value of 1. The liquor thus acidified was filtered through a vacuum unit, the acidified liquor being able to be filtered through all sorts of tested paper filters without any deposits on the filter.

The same original black liquor was first set to 35° C. and then brought to pH 1 with the help of sulphuric acid. The resultant mixture was so viscous that in a filtration unit all types of filter papers were almost immediately clogged. The centrifuging of the mixture produced no separation of solids.

A soluble organic polyelectrolyte was used in order to coagulate the acidified mixture. The latter was then filtered through ash-free filter paper, the precipitate thus being separated. After air drying the solids thus collected were black and had an ash content of 25% which was presumably attributable to inorganic salts which remained in the precipitate.

In a further experiment, 200 ml of the original black liquor was acidified at a temperature of 35° C. to pH 1, after that heated up to 85° C. and mixed for 10 min, the temperature being kept at 85° C. The resultant slurry precipitated was cooled down to 30° C. and filtered through a vacuum filtration unit with a paper filter. Filtration took roughly 5 minutes. The resultant filter cake was washed with 50 ml of water, the water taking 4 minutes to pass through the filter. The filter cake was dried at 80° C. and yielded 8 g of a yellow substance with a measured ash content of 1%. The quantity of carboxyl groups, measured in milliequivalents amounted to 1 milliequivalent per g of solid matter. The quantity of organic substances in the filtrate, measured as COD, amounted to 68 g/l.

The yellow material from the last experiment was retained in this form for applications with regard to the method according to this invention.

### Example 4

### Production of Sulphur-Free, Carboxylated Lignin From Flax and Hemp Straw Black Liquor by Means of a Precipitation and Filtration Process

Black liquor from flax-straw and hemp-straw pulp, which was produced using the soda AQ process from example 2, was used. The liquor had an organic-substance content, measured as COD (chemical oxygen demand) of 110 g/l, a total basicity, expressed as g/l of NaOH, of 18.3 g/l, a point of change of the acid titration curve at pH 3.8 and the maximum viscosity with progressive acidifying at pH 2.5. The temperature of the black liquor was set to 30° C. and by the addition of gaseous carbon dioxide brought to below pH 7, followed by further acidifying to pH 1 by means of sulphuric acid, the temperature being maintained. The mixture was then slowly heated up to 85° C. and then kept at the same temperature for 10 min. The resultant slurry precipitated was cooled down to 30° C. and filtered through a paper filter under vacuum.

The filtration lasted roughly 5 minutes for each 200 ml of slurry, a new filter being used each time. Each filter cake was washed with 50 ml of water, the water taking 5 minutes to pass through the filter. The filter cakes were collected and dried at 80° C. and yielded a yellow substance with a total weight of 65 g, an ash content of 0.8% and carboxyl group content, measured in milliequivalents, of 1.2 milliequivalents per g of solids. The quantity of organic substances in the filtrate, measured as COD, amounted to 68 g/l.

The carboxylated lignin derivative in the form of a dried yellow powder was retained for applications with regard to the method according to this invention.

### Example 5

### Production of a Solution From Sulphur-Free Carboxylated Lignin Which Originates From Wheat Straw

1020 g of water and 38 g of NaOH were added to 500 g of a dry filter cake of carboxylated lignin which was produced as in example 3. This mixture was mixed for 24 h at room temperature and vacuum filtered through cellulose fibre filters. At room temperature, the filtrate had a solids content of 30%, a pH of 10 and a viscosity of 85 cPs. In this form the solution was retained for the application concerning the method according to this invention.

### Example 6

### Production of a Solution of Sulphur-Free Carboxylated Lignin That Originates From Flax and Hemp Straw

1100 g of water and 45 g of NaOH were added to 500 g of a dry filter cake of carboxylated lignin which was produced as in example 4. This mixture was mixed at room temperature for 48 hrs and vacuum filtered through cellulose fibre filters. At room temperature the filtrate had a solids content of 31%, a pH of 10.3 and a viscosity of 98 cPs. The solution was retained in this form for the application regarding the process according to this invention.

### Example 7

### Mixture of a Sulphur-Free, Carboxylated Lignin Derivative With Sodium Polyasparaginate as the Complexing Agent

59 g of a 38% Na polyasparaginate solution was added to 500 g of the solution of carboxylated lignin from example 5 (the Na polyasparaginate was a development product, number TP OC 2401 from Bayer AG, Leverkusen). The resultant mixture was mixed for one hour and filtered through a cellulose fibre filter. At room temperature the pH value was 9.5 and the viscosity was 78 cPs. The solution was retained in this form for the application in the method according to this invention.

### Example 8

### Mixture of a Sulphur-Free, Carboxylated Lignin Derivative With Sodium Asparate as the Complexing Agent

59 g of a 38% Na polyasparaginate solution was added to 500 g of the solution of carboxylated lignin from example 6 (the Na polyasparaginate was a development product, number TP OC 2401 of Bayer AG, Leverkusen). The resultant mixture was mixed for one hour and filtered through a cellulose fibre filter. At room temperature the pH value was 9.9 and the viscosity 90 cps. The solution was retained in this form for the application regarding the method according to this invention.

### Example 9

### Production of Sulphur-Free Lignin From Wood by a Tetramethyl Ammonium Hydroxide Pulp Process and Subsequent Precipitation

Wood chips were boiled at high temperature and under pressure in a solution of tetramethyl ammonium hydroxide in a pressure reactor. During cooling-down to room temperature the resultant pulp was washed with a minimal quantity of water; the black liquor was retained for further treatment in order to separate off a fraction of lignin derivatives. The black liquor was acidified with sulphuric acid and the temperature was raised to a point at which the precipitated lignin could be filtered. After filtration the lignin yielded a yellowish, moist cake. The moist lignin was dispersed in water; sodium hydroxide was added up to a pH value of 9.5, after some time a solution of the lignin forming which was filtered. The resultant solution of sulphur-free lignin had a solids content of 15% and was intended for the application regarding the method according to this invention.

### Application examples

### Example 10

### Reduction of Slime and Deposits in a System in Which Paper Machine White Water is Circulated, by Treatment With a Sulphur-Free Carboxylated Lignin Derivative

A number of 51 containers were used in order to check a sulphur-free, carboxylated lignin derivative, commercially available lignosulphonates and biocides with regard to their effect on the formation of slime and deposits.

The white water originated from the white water I circuit of a paper machine which produces wood-free paper with carbonate filler, used painted rejects and starch for the production of the stock and runs at a slightly alkaline pH. The white water was sampled after a re-start, before any slime combating systems were used. The white water had a solids content of 4.6 g/l, of which 3.0 g/l were determined to be fillers (CaCO₃). Seven tanks were filled with the white water up to the 5-litre mark, the magnetic agitators were switched on, as well as the thermostatically controlled heating systems which were set to 36° C. Weighed slime measuring boards were fully immersed in the liquid. The microbial activity in the test tanks was checked by plating out on bacterial count-Agar from the company of Merck. The additions to the slime check to be compared were diluted in a suitable way and a certain quantity was added to each test tank. After 72 hours the bacterial counts were determined again and the measuring boards covered in slime were carefully weighed. After the measurements after 72 hours the slime measur8ing boards were dried at 105° C. and again weighed in order to determine the dry weight of the stored material. The dried material was then removed from the boards and incinerated in a furnace in porcelain crucibles at 550° C. The ash was weighed back in order to measure the percentage of mineral substances in the original deposits. The whole experiment was repeated with various concentrations of the individual slime combating products.

Tested products:

1) No product (control)

2) Solution of sulphur-free, carboxylated lignin from example 5 (carboxylated lignin from wheat straw)

3) Solution of sulphur-free, carboxylated lignin from example 6 (carboxylated lignin from flax/hemp straw)

4) Type A, commercial lignin sulphonate solution as is used for slime combating in paper machines

5) Type B, commercial lignin sulphonate solution as supplied in the construction industry as a dispersing agent

6) Commercial biocide solution which contains methylene-bis-thiocyanate

7) Commercial biocide solution which contains a quaternary ammonium salt

As can be seen from Table 1, those test approaches in which the products with sulphur-free, carboxylated lignin from the examples 5 and 6 were used, yielded the lowest dry weight of the deposits. What was, seen from the practical point of view, even more significant was the fact that these approaches reveal even the slightest solids content in the deposits themselves. It is an empirical value that such deposits with a low solids content can be dispersed considerably more easily with the help of the flow and pose less of a threat to the performance of the paper machine

The checking and the two test approaches which were treated with biocides, had the lowest ash content. This suggested that in these deposits there was a greater growth of organisms and/or a more marked colony formation taking place in situ and not a progressive depositing of all of the solid substances present. Slimes which consist of colonies of micro-organisms, can, because of the difficulties encountered in dispersing them, pose a serious problem for paper production.

The results are summarised in Table 1.

TABLE 1

| | | | | | | | | | |
|---|---|---|---|---|---|---|---|---|---|
| | | | | | | | | | Ash content |
| | | Addition | Bacterial | Bacterial | | | | Solids content | in deposits |
| | | (active) | count at | count after | Moist | Dry | Solids | in deposits | in % of the |
| | | ppm per | beginning | 72 hrs | weight of the | weight of the | content in | in % of | solids content |
| Sample | Type | volume | CFU/ml | CFU/ml | deposit in g | deposit in g | deposit in % | the control | of the control |

| | | | | | | | | | |
|---|---|---|---|---|---|---|---|---|---|
| 1) | No addition | 0 | 7.8 × 10⁵ | 5.3 × 10⁶ | 2.045 | 0.450 | 22 | 100 | 10 |
| 2) | Carboxylated | 50 | ″ | 7.2 × 10⁵ | 0.770 | 0.077 | 10 | 17 | 21 |
| | lignin from | | | | | | | | |
| | example 5 | | | | | | | | |
| 3) | Carboxylated | 50 | ″ | 6.4 × 10⁵ | 1.103 | 0.072 | 7 | 16 | 21 |
| | lignin from | | | | | | | | |
| | example 6 | | | | | | | | |
| 4) | Lignosulphonate | 50 | ″ | 6.8 × 10⁵ | 0.720 | 0.086 | 12 | 19 | 26 |
| | A | | | | | | | | |
| 5) | Lignosulphonate | 50 | ″ | 4.0 × 10⁶ | 1.814 | 0.380 | 21 | 84 | 22 |
| | B | | | | | | | | |
| 6) | MBT biocide | 50 | ″ | 2.7 × 10⁴ | 0.505 | 0.081 | 16 | 18 | 12 |
| 7) | Quat. Biocide | 50 | ″ | 5.8 × 10⁴ | 0.582 | 0.099 | 17 | 22 | 14 |

### Example 11

### Reduction of Slime and Deposits in a System in Which Paper Machine White Water is Circulated, by Treatment With a Mixture of a Carboxylated Lignin Derivative and a Complexing Agent

The same test record was followed as in example 10, however using the following products:

Tested products:

1) No product (check)

2) Solution of sulphur-free, carboxylated lignin/Na polyasparaginate from example 7 (carboxylated lignin from wheat straw)

3) Solution of sulphur-free, carboxylated lignin/Na polyasparaginate from example 8 (carboxylated lignin from flax/hemp straw)

4) Solution of sulphur-free, carboxylated lignin from example 5 (carboxylated lignin from wheat straw)

5) Solution of sulphur-free, carboxylated lignin from example 6 (carboxylated lignin from flax/hemp straw)

6) Type A, commercial lignin sulphonate solution as is used for slime combating in paper machines.

The white water used had a solids content of 4.1 g/l, of which 2.9 g/l were carbonate filler.

As can be seen from the solids content of the deposits the products with sulphur-free lignin, if they were used in combination with a complexing agent, even yielded an even lower dry weight in the deposits, the deposits being very easy to disperse with low flow.

The results are summarised in Table 2.

TABLE 2

| | | | | | | | | | Ash content |
|---|---|---|---|---|---|---|---|---|---|
| | | Addition | Bacterial | Bacterial | | | | Solids content | in deposits |
| | | (active) | count at | count after | Moist | Dry | Solids | in deposits | in % of the |
| | | ppm per | beginning | 72 hrs | weight of the | weight of the | content in | in % of | solids content |
| Sample | Type | volume | CFU/ml | CFU/ml | deposit in g | deposit in g | deposit in % | the control | of the control |
| 1) | No addition | 0 | 1.1 × 10⁶ | 6.2 × 10⁶ | 1.892 | 0.359 | 19 | 100 | 12 |
| 2) | Carb. | 50 | ″ | 5.4 × 10⁶ | 1.301 | 0.119 | 9 | 33 | 25 |
| | Lignin/ | | | | | | | | |
| | polyasparaginate - | | | | | | | | |
| | mix. from | | | | | | | | |
| | example 7 | | | | | | | | |
| 3) | Carb. | 50 | ″ | 5.1 × 10⁶ | 1.285 | 0.120 | 9 | 33 | 27 |
| | Lignin/ | | | | | | | | |
| | polyasparaginate - | | | | | | | | |
| | mix. from | | | | | | | | |
| | example 8 | | | | | | | | |
| 4) | Carboxylated | 50 | ″ | 5.0 × 10⁶ | 1.217 | 0.139 | 11 | 39 | 23 |
| | lignin from | | | | | | | | |
| | example 5 | | | | | | | | |
| 5) | Carboxylated | 50 | ″ | 5.7 ×10⁶ | 1.194 | 0.116 | 10 | 32 | 25 |
| | lignin from | | | | | | | | |
| | example 6 | | | | | | | | |
| 6) | Lignosulphonate/ | 50 | ″ | 6.0 × 10⁶ | 1.255 | 0.137 | 11 | 38 | 22 |
| | complexing | | | | | | | | |
| | agents - mix. | | | | | | | | |

### Example 12

### Reduction of Slime and Deposits in a System in Which Paper Machine White Water is Circulated by Treatment With a Mixture of a Sulphur-Free, Carboxylated Lignin Derivative and With the Addition of a Small Quantity of Biocide

The same test record as in example 10 was followed, however using the following products

Tested products:

1) No product (control)

2) Solution of sulphur-free, carboxylated lignin from example 6 (carboxylated lignin from flax/hemp straw)

3) Solution of sulphur-free, carboxylated lignin from example 6 plus the biocide 2-bromium-2-nitropropane-1,3-diol

4) 2-bromium-2-nitropropane-1,3-diol

The white water used had a solids content of 4.7 g/l, of which 3.2 g/l were carbonate filler.

In this experiment it turned out that a combination of sulphur-free, carboxylated lignin from example 6 with a small quantity of the biocide yields the lowest dry weight of the deposits.

The results are summarised in Table 3.

TABLE 3

| | | | | | | | | | Ash content |
|---|---|---|---|---|---|---|---|---|---|
| | | Addition | Bacterial | Bacterial | | | | Solids content | in deposits |
| | | (active) | count at | count after | Moist | Dry | Solids | in deposits | in % of the |
| | | ppm per | beginning | 72 hrs | weight of the | weight of the | content in | in % of | solids content |
| Sample | Type | volume | CFU/ml | CFU/ml | deposit in g | deposit in g | deposit in % | the control | of the control |
| 1) | No addition | 0 | 4.2 × 10⁵ | 6.9 × 10⁶ | 3.220 | 0.547 | 17 | 100 | 9 |
| 2) | Carboxylated | 70 | ″ | 1.5 × 10⁶ | 1.318 | 0.118 | 9 | 22 | 20 |
| | lignin from | | | | | | | | |
| | example 6 | | | | | | | | |
| 3) | Carboxylated | 50 + 20 | ″ | 6.7 × 10⁵ | 1.144 | 0.101 | 9 | 19 | 23 |
| | lignin from | | | | | | | | |
| | example 6 + | | | | | | | | |
| | bromiumnitro- | | | | | | | | |
| | propane diol | | | | | | | | |
| 4) | bromiumnitro- | 70 | ″ | 8.1 × 10⁴ | 1.020 | 1.026 | 12 | 23 | 17 |
| | propane diol | | | | | | | | |

### Example 13

### Reduction of Slime and Deposits in Spray Water Regions of a System, in Which Paper Machine White Water is Circulated by Treatment of the Spray Water With a Mixture of a Sulphur-Free, Carboxylated Lignin Derivative and a Complexing Agent

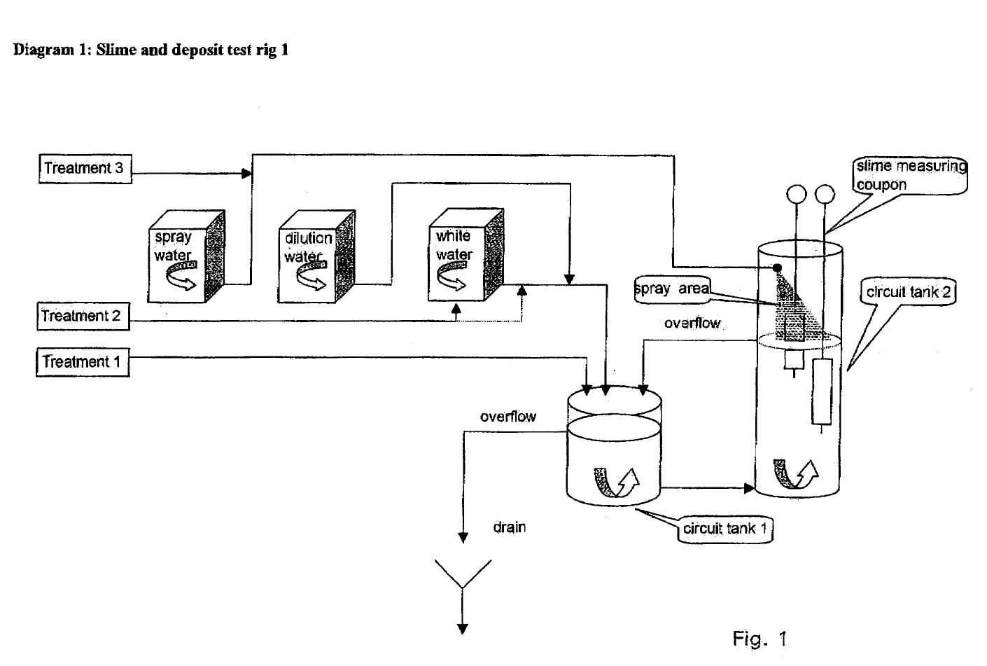

A test unit in accordance with the diagram of FIG. 1 was used, of which parts and typical dimensions are explained below:

Circuit tank 1: 10 l working volume, pumps to circuit tank 2, at about 100 l/h, where flow velocity and flow are large enough to keep solids in the white water in suspension. The tank has a thermostat heater/temperature controls.

Circuit tank 2 (slime measuring tank): 10 l working volume, in circuit with tank 1, with overflow back into the latter tank. The tank is equipped with one each or several each wholly or half immersed slime measuring boards which consist of sheets of thin stainless steel and are suspended from a fine wire. The tank has a free volume above the liquid level which permits generating a closed, half-closed or open atmosphere. In this chamber there is a lighting system and a spray nozzle, the water sprayed during use hits the exposed (not submerged) parts of the slime measuring board(s).

White water tank: Approx. 200 l capacity, has a mixer in order to keep solids in the white water in suspension. White water can, for example, be dosed at a rate of 5 l/h into tank 1 with a small diaphragm pump.

Dilution water tank: Approx. 200 l capacity. Can be filled with fresh water or a selected process water.

Spray water tank: Approx. 200 l capacity. Can be filled with a selected process water or with fresh water, or fresh water can be supplied direct from the water mains.

Spray region: The free volume in circuit tank 2 simulates a spray region of a paper machine in which here the humidity, form of the spray, spray liquid, treatment of the spray liquid, lighting etc. can be influenced. That makes it possible to measure tendencies in the formation of slime and deposits and to examine the particular treatment.

Slime measuring boards: Thin sheets of special steel which are fastened to a fine wire; all of the boards are numbered and their weight is known. Can be removed for weighing or drying and weighing in order to determine the growth of slime.

Treatment systems: The various treatment additives are, if necessary, diluted suitably and metered through calibrated peristaltic pumps at the points required.

Treatment 1: Suitable for the addition of sulphur-free lignin or its derivatives, or mixtures thereof with complexing agents. The metering point is located on circuit tank 1.

Treatment 2: Additional supplementary tank, suitable for biocide, periodically dosed either into the white water tank or into the line leading to circuit tank 1.

Treatment 3: Treatment for spray water. Can be carried out continuously at periodic intervals etc. The treatment can consist of sulphur-free lignin or its derivatives, or of a mixture of them with complexing agents, biocides, hydrogen peroxide etc.

Test Record

White water originated from the white water I circuit of a paper machine which produces wood-free paper with carbonate filler, uses painted rejects and starch for the production of the stock and runs at a slightly alkaline pH. The white water was sampled after a re-start-up, before any slime combating systems were used. The white water had a solids content of 3.8 g/l, of which 2.8 g/l were determined as filler (CaCO₃). The white water tank (unheated), circuit tank 1 and circuit tank 2 were filled with white water. The spray water tank was filled with tap water from the water mains. The agitators for the white water and spray water tanks were switched on, likewise the pump between tank 1 and tank 2 which were set to a flow velocity of approximately 100 litres per hour. A pump which pumped the white water from the storage tank into circuit tank 1 was switched on and set to 1.5 l/h. A corresponding volume therefore flowed from the overflow of circuit tank 1 into the drain. The heating system for the circuit was set to a temperature of 36° C.

Spray water from a system consisting of a high-pressure pump and an aerosol nozzle was set in such a way that at a velocity of 1 l/h a fine aerosol spray onto the upper part of the circuit tank was produced. The corresponding volume flowed, in turn, through the overflow of circuit tank 1 into the drain.

After 5 days under these conditions the partially immersed slime measuring boards were removed from the spray region of circuit tank 2 for analysis.

At and directly above the boundary between the air and the white water a pink-coloured deposit formed. Microscopic analysis showed that the deposit consisted of an interwoven mass of filamentous bacteria. Under polarised light it could be seen that there was very little filler present in the slime (fillers on a CaCO₃ basis appear as bright white dots). The growth of the slime extended to down to one centimetre below the surface of the white water where a general transition to a white deposit took place. This white deposit extended evenly over the rest of the submerged surface of the test board. Under the microscope it could be seen that the deposits under the water contained a significant percentage of fillers. The slime above the surface of the water was also, like that under the surface, removed for an analysis of the solids content.

Treatment of the Spray Water

In a second experiment the test unit was set up, filled and started up under the same conditions as above, however the spray water was treated. A solution—with an active content of 0.1%—of the mixture of carboxylated lignin and polyasparaginate from example 6 was produced with tap water. This was metered at 50 ml/h continuously into the suction side of the spray water pump, which resulted in a treatment level of 50 ppm in the spray water.

After 5 days under these conditions the partially immersed slime measuring boards were removed from the spray region of circuit tank 2 for analysis.

At and directly above the boundary between air and white water a slightly whitish coloured deposit was present. Microscopic analysis showed that the deposit at and above the air/water boundary contained almost no filamentous bacteria and that some filler was present. Under water a white deposit covered the board homogeneously. The deposits were analysed in the same way as those from the test without treatment.

The results are summarised in Table 4.

TABLE 4

| | Addition | | | |
|---|---|---|---|---|
| | (active) | | | |
| | in ppm | | | Filler |
| | per | Solids | Filamentous | (optical |
| Type | volume | content | forms | microscope) |
| Slime above surface | 0 | 19 | Many | Not many, |
| of water - | | | | at isolated |
| untreated spray | | | | points |
| water | | | | |
| Slime above the | 50 | 14 | Very little | A little |
| surface of the | | | | |
| water - treated | | | | |
| spray water | | | | |
| Slime below the | 0 | 15 | Almost none | Evenly |
| surface of the | | | | distributed |
| water - untreated | | | | |
| spray water | | | | |
| Slime below the | 50 | 9 | None | Evenly |
| surface of the | | | | distributed |
| water - treated | | | | |
| spray water | | | | |

In that region where untreated spray water impinged on the measuring board, the deposits above the surface of the water consisted of filamentous bacteria forming colonies and they resembled the slime which as is well known produces problems in paper production because the poor dispersibility of the deposits can lead to stains in the paper. The deposits at and above the surface of the water from the experiment in which the treated spray water was used did not show any such colonies of filamentous bacteria and were easier to disperse.

The deposits on the measuring board below the surface of the water were, in the experiment with treated spray water, easier to disperse than those from the experiment without treatment; that suggested that treatment of the spray water with a solution of the mixture of sulphur-free, carboxylated lignin and polyasparaginate from example 6 exerts a positive effect on the reduction of deposits on surfaces below the surface of the water in the circuit.

### Example 14

### Reduction of Slime and Deposits in a System in Which Paper Machine White Water is Circulated, by Treatment With Sulphur-Free Lignin

A similar process to the one in example 10 was used in order to compare sulphur-free lignin and a lignosulphonate which is commercially available and is intended to be a means for the checking of deposits with regard to their effect on the formation of slime and deposits.

White water which originated from the white water I circuit of a paper machine which produced paper with a carbonate filler and ran at lightly alkaline pH, was used for filling the test tanks. The white water was sampled after a re-start-up, before any slime-combating systems were used. The white water had a solids content of 3.5 g/l, of which 2.6 g were determined as filler (CaCO₃).

Three tanks were filled up to the 5-litre mark with the white water, the magnetic agitators were switched on, likewise the thermostat-controlled heating systems which were set to 36° C. Weighed slime measuring boards were immersed completely in the liquid. The microbial activity in the test tanks was checked by plating-out to bacterial count-Agar of the company of Merck. The products to be compared were diluted in a suitable way and a certain amount was added to every test tank. After 72 hours the bacterial counts were determined again. The measuring boards were carefully removed. If no water dripped off any longer, the slime was completely scraped off and transferred to glass beakers. Sterile water was added and the material out of the deposits was shaken up, until there were no more lumps or colonies of micro-organisms visible under the microscope and everything was homogeneously dispersed. The resultant suspensions were plated-out to determine the bacterial counts. This was used as a measure of how many micro-organisms were present in the deposits on the measuring boards.

Tested Points:

1) No product (control)

2) Solution of sulphur-free lignin from example 9 (lignin from wood)

3) Type A, commercial lignosulphonate solution as is used for slime-combating in paper machines

The results are summarised in Table 5.

TABLE 5

| | | | | Bacterial |
|---|---|---|---|---|
| | | | | count from |
| | | | | the |
| | | | | dispersed |
| | | Bacterial | | deposits |
| | | count in the | Bacterial | from the |
| | Addition | white water | count in | measuring |
| | (active) | at the | white water | boards |
| | in ppm per | start of | after 72 | after 72 |
| Treatment agent | volume | CFU/ml | hrs CFU/ml | hrs CFU/ml |
| None | 0 | 3.6 × 10⁴ | 5.2 × 10⁵ | 7.7 × 10⁴ |
| Sulphur-free | 50 | ″ | 4.0 × 10⁵ | 1.6 × 10³ |
| lignin from | | | | |
| example 9 | | | | |
| Lignsulphonate A | 50 | ″ | 4.5 × 10⁵ | 2.1 × 10³ |

As can be seen from Table 5, the sulphur-free lignin product from example 9 and the commercial lignin product yield an at least similar reduction of the bacterial count present in the deposits in comparison with the control.

Finally it can thus be stated that the method according to the invention can be successfully used in the white water circuit of a paper machine, at the water spray of a paper machine at the place of contact with the circuit water, but also to a certain extent for the preservation of solutions of auxiliary materials used in paper production, such as starch, slurries of raw materials, for example fillers, or paper rejects. The application for preservation in the last two cases mentioned is particularly advantageous because then the active ingredient is already introduced into the system with the solutions, slurries etc.

1. Method for the reduction of the formation of slime and deposits in closed and/or semi-closed aqueous or acquiferous systems, sulphur-free lignin or a derivative thereof being added to the system in a quantity proportional to the quantity of substances present in the water causing slime and deposits.

2. Method according to claim 1, characterised by the fact that the derivatives of the sulphur-free lignin are chosen as being of carboxylated, phosphonated or nitrated sulphur-free lignin.

3. Method according to claim 1 or 2, characterised by the fact that one uses carboxylated, sulphur-free lignin.

4. Method according to one of the claims 1 to 3, characterised by the fact that the sulphur-free lignin or its derivative is obtained from annual plants, in particular straw, hemp or flax.

5. Method according to claim 4, characterised by the fact that the sulphur-free lignin or its derivative is obtained from annual plants using the soda process or the soda anthraquinone process for the production of pulp.

6. Method according to claim 4 or 5, characterised by the fact that the sulphur-free lignin or its derivative is obtained from the alkaline solution of the soda process or of the soda anthraquinone process by acidification of the alkaline solution and treatment of the acidified solution in selected temperature ranges and separation of the precipitate from the liquid phase.

7. Method according to claim 6, characterised by the fact that a first temperature range is between 30° C. and 40° C. and a second temperature range is between 80° C. and 90° C.

8. Method according to one of the claims 1 to 3, characterised by the fact that the sulphur-free lignin or its derivative is obtained from wood.

9. Method according to claim 8, characterised by the fact that the sulphur-free lignin or its derivative is obtained from wood, using the tetramethyl ammonium hydroxide process.

10. Method according to one of the claims 1 to 9, characterised by the fact that the sulphur-free lignin or the lignin derivative, based on the active product content, is added to the system at a rate of 0.1 g to 1.8 g per m³ of circuit water.

11. Method according to one of the claims 1 to 10, characterised by the fact that the sulphur-free lignin or its derivative is use together with a complexing agent.

12. Method according to claim 11, characterised by the fact that the complexing agent is polyasparaginate.

13. Method according to claim 11 or 12, characterised by the fact that the quantity if active complexing agent, calculated as a percentage share of the sulphur-free lignin or its derivative, is 5% to 25%, preferably 10 to 20%, more preferably 15%.

14. Method according to one of the claims 1 to 10, characterised by the fact that one uses the sulphur-free lignin or its derivative together with a biocide.

15. Method according to claims 14, characterised by the fact that the biocide is selected from among 3,5-dimethyl-tetrahydro-1,3,5-thiadiazin-2-thion, methylene-bis-thiocyanate, carbamatene, 2,2-dibromium-3-nitrile-propionamide and 2-bromium-2-nitropropane-1,3-diol.

16. Method according to claim 15, characterised by the fact that the biocide is 2-bromium-2-nitropropane-1,3-diol.

17. Method according to one of the claims 14 to 16, characterised by the fact that the biocide is added at a rate of 10 to 180 g/t or more, in particular 50 to 100 g/t, based on the solids content of the fluid containing the paper stock, preferably 70 g/t.

18. Method according to one of the claims 1 to 10, characterised by the fact that one uses the sulphur-free lignin or its derivative together with a complexing agent and a biocide.

19. Method according to claim 18, characterised by the fact that the complexing agent and the biocide comply with the definitions and the statements of the claims 11 to 17.

20. Method according to one of the preceding claims, characterised by the fact that one adds the sulphur-free lignin or its derivative to the white water circuit of a paper machine.

21. Method according to one of the claims 1 to 19 or 20, characterised by the fact that one adds the sulphur-free lignin or its derivative to the spray water of a paper machine at the point of contact with the circuit water.

22. Method according to one of the claims 1 to 19, characterised by the fact that one adds the sulphur-free lignin or its derivative to the process water for the storage of the latter, in particular for its temporary storage.

23. Method according to one of the claims 1 to 19, characterised by the fact that one adds the sulphur-free lignin or its derivative to the solutions of auxiliary materials, slurries of raw materials or paper rejects used in paper production.
